(19) United States
(12) Patent Application Publication
(10) Pub. No.: US 2013/0253249 A1
(43) Pub. Date: Sep. 26, 2013

(54) DEHYDROGENATION PROCESS

(71) Applicant: Xavier Elie Baucherel, Cleveland (GB)

(72) Inventor: Xavier Elie Baucherel, Cleveland (GB)

(73) Assignee: JOHNSON MATTHEY PUBLIC LIMITED COMPANY, London (GB)

(21) Appl. No.: 13/991,311

(22) PCT Filed: Dec. 2, 2011
(86) PCT No.: PCT/GB11/52382

§ 371 Date: Jun. 3, 2013

(30) Foreign Application Priority Data
Dec. 3, 2010 (GB) 102050.1

(51) Int. Cl. C07C 5/333

(52) U.S. Cl. CPC C07C 5/3332
USPC 585/661; 502/180; 502/182; 502/184; 502/185

(57) ABSTRACT

A method of dehydrogenating a hydrocarbon, especially an alkane, to form an unsaturated compound, especially an alkene, includes contacting the alkane with a catalyst including a form of carbon which is catalytically active for the dehydrogenation reaction. The catalyst may be formed by passing a hydrocarbon over a catalyst precursor at an elevated temperature for sufficient time to form the active carbon phase, characterized in that the catalyst precursor includes less than 0.1% of a transition metal.

The present invention concerns processes for the dehydrogenation of hydrocarbon compounds, and catalysts used for such processes.

Catalytic dehydrogenation of hydrocarbon chains, especially alkanes, is an important process commercially for the production of unsaturated compounds. In particular the production of alkenes such as propene and butenes by dehydrogenation of the corresponding alkanes, i.e. propane and butane, form an important source of feedstocks for the manufacture of polyolefins and other products.

Processes for the dehydrogenation of alkanes are well known and widely used in industry. Non-oxidative dehydrogenation processes may be conducted using transition metal catalysts such as vanadia or chromia at temperatures of up to about 550° C. These catalysts deactivate rapidly under reaction conditions due to the formation of carbon deposits on the catalyst. The catalyst is periodically regenerated by burning off the carbon in an oxidation step. For example, GB-A-837 707 describes dehydrogenation of hydrocarbons employing a regenerable chromia catalyst wherein part of the chromia is oxidised to the hexavalent state during the oxidative regeneration process. The description indicates that the heat of combustion of the by-product carbon during the regeneration step can supply the heat required for the dehydrogenation reaction and that the reduction of the hexavalent chromium compound, which occurs during the reaction stage, can supplement the heat. This type of process is still widely used for the production of propene and butene but the requirement to regenerate the catalyst, typically after 20-30 minutes online, increases the cost and complexity of the process and the plant required. U.S. Pat. No. 5,087,792 describes an alternative process for the dehydrogenation of a hydrocarbon selected from the group consisting of propane and butane using a catalyst comprising platinum and a carrier material wherein the spent catalyst is reconditioned in a regeneration zone that uses, in the following order, a combustion zone, a drying zone and a metal re-dispersion zone to remove coke and recondition catalyst particles.

In U.S. Pat. No. 5,220,092 and EP-A-0556489, alkanes are dehydrogenated by contacting them with a catalyst containing vanadia on a support at elevated temperature for less than 4 seconds; a contact time of 0.02 to 2 seconds is said to give very good results. The alkanes are fed to the catalyst as short pulses interrupting a continuous flow of argon. A continuous regeneration of the catalyst for removal of coke, similar to the regeneration carried out in a fluidised catalytic cracking reaction, is preferred.

US-A-2008/0071124 describes the use of a supported nanocarbon catalyst for the oxidative dehydrogenation of alkylaromatics, alkenes and alkanes in the gas phase. This reference does not, however, describe or suggest that carbon nanostructures are stable and catalytically active for dehydrogenation reactions under non-oxidising conditions, i.e. in the absence of an oxygen-containing gas.

Processes for the oxidative dehydrogenation of alkanes are also practised using various metal oxide catalysts and mixed metal oxides. Such processes have the disadvantage that the oxidising conditions may cause the formation of oxygenated by-products such as alcohols, aldehydes, carbon oxides and also convert at least some of the produced hydrogen to water. There is a need for improved dehydrogenation processes, in particular for the production of lower alkenes such as propene and butene.

According to the invention we provide a process for carrying out a chemical reaction comprising the step of passing a feed stream containing at least one reactant compound over a catalyst comprising a catalytically active carbon phase, wherein said catalyst is formed by passing a hydrocarbon-containing gas over a catalyst precursor at an elevated temperature for sufficient time to form the active carbon phase, characterised in that said catalyst precursor comprises less than 0.1%, especially less than 0.07% of a transition metal. In particular, the catalyst precursor preferably comprises less than 0.1%, preferably less than 0.07%, particularly less than 0.05% (and especially <0.01%) by weight of V, Cr, Mn, Fe, Co, Mo, Ni, Au, Pt, Pd, Ru or Rh. The catalyst precursor preferably comprises these elements only as impurities. In the case of a carbon support, the amount of metal impurities may exceed 0.1%, and in some cases may be as high as 0.5%. These materials are not currently preferred. The amount of these elements in the catalyst precursor is measured by X-Ray fluorescence spectroscopy.

According to a second aspect of the invention we provide a process for carrying out a chemical reaction comprising the step of passing a feed stream containing at least one reactant compound over a catalyst comprising a catalytically active carbon phase, wherein said catalyst is formed by passing a hydrocarbon-containing gas over a catalyst precursor at an elevated temperature for sufficient time to form the active carbon phase, characterised in that said catalyst precursor consists of a material selected from the group consisting of alumina, silica, magnesia, zirconia, titania, ceria, silica-alumina, carbon and mixtures of these materials.

According to a further aspect of the invention we provide a process for dehydrogenating a hydrocarbon comprising the step of contacting a catalyst precursor consisting of a material selected from the group consisting of alumina, silica, magnesia, zirconia, titania, ceria, silica-alumina, carbon and mixtures of these materials and containing <0.1%, especially <0.05% by weight of V, Cr, Mn, Fe, Co, Mo, Ni, Au, Pt, Pd, Ru or Rh with a hydrocarbon at a temperature greater than 600° C. for a period of at least one hour.

According to a further aspect of the invention we provide a method of forming a catalyst comprising a form of carbon which is active for the dehydrogenation of alkanes, by contacting a catalyst precursor comprising less than 0.1%, especially less than 0.07% of a transition metal, especially a metal selected from the group consisting of V, Cr, Mn, Fe, Co, Mo, Ni, Au, Pt, Pd, Ru and Rh, with a hydrocarbon at a temperature of at least, and preferably greater than, 600° C.

According to an alternative aspect of the invention we provide a method of forming a catalyst comprising a form of carbon which is active for the dehydrogenation of alkanes, by contacting a catalyst precursor with a hydrocarbon at a temperature of at least, and preferably greater than, 600° C., characterised in that said catalyst precursor consists of a material selected from the group consisting of alumina, silica, magnesia, zirconia, titania, ceria, silica-alumina, carbon black and mixtures of these materials.

The hydrocarbon is conveniently an alkane. In a preferred form of the process the hydrocarbon used to form the active catalyst comprises the alkane contained in a feed stream for a dehydrogenation reaction. The catalyst including the active carbon phase may be formed ex-situ or in-situ in the reactor in which it is to be used as a catalyst. It is a particular benefit that the catalyst may be formed in the reactor used for dehydrogenation by contact of a catalyst precursor with a hydrocarbon at a suitable temperature which is preferably at least 600° C. and then used to catalyse the dehydrogenation of said alkane.

A significant difference between the process of the invention and dehydrogenation processes known in the art is that the coke deposits formed in the dehydrogenation reaction are not removed through oxidation or other catalyst regeneration steps. In the process of the invention the coke formed in the reaction remains on the catalyst within the reactor. The coke formed during the reaction is believed to be catalytically active, i.e. it contains catalytically active carbon species. Therefore the dehydrogenation process of the invention is operated in the absence of a catalyst regeneration step. Prior art catalyst regeneration usually involves oxidation of the coke deposited on the catalyst and this is typically carried out frequently, possibly more than once per hour of reaction time. It is a feature of the present invention that the process is preferably operated for more than 12 hours, especially more than 24 hours without catalyst regeneration or removal of the coke or carbon deposits formed.

The chemical reaction is preferably a dehydrogenation reaction and the reactant is preferably a hydrocarbon, in particular an alkane.

The elevated temperature is preferably at least 600° C., particularly between 600° C. and 700° C., and most preferably in the range from 620-700° C.

The feed stream containing the hydrocarbon is contacted with the catalyst precursor for sufficient time at the elevated temperature for carbon to form on the catalyst surface. Preferably sufficient carbon is formed on the catalyst so that at least 3%, more preferably at least 5% of the catalyst, by weight, comprises carbon formed by reaction of a hydrocarbon containing feed stream with the catalyst at said elevated temperature. The process is preferably operated by contacting said feed stream with said catalyst or precursor at said elevated temperature for at least 1 hour, more preferably at least 3 hours, especially at least 6 hours. This contact enables an active phase of carbon to form on the catalyst.

We have found that the active carbon forms effectively during an activation phase of the process when the catalyst precursor is contacted with the hydrocarbon at a temperature in the range 600-750° C. for at least 30 minutes, more preferably at least 1 hour and especially at least 3 hours. It is preferred to contact the catalyst precursor with the hydrocarbon at a temperature between 680 and 750° C. during this activation phase, more preferably between 680 and 725° C. Following the activation phase, during which it is believed that the active carbon species are formed, the dehydrogenation reaction may then proceed at a different temperature, preferably a lower temperature, for example from 600-700° C., especially 600-670° C.

The active carbon catalyst may be formed by passing a hydrocarbon over a precursor material consisting essentially of a material selected from the group consisting of alumina, silica, magnesia, zirconia, titania, ceria, silica-alumina, carbon or mixtures of these materials at a temperature of at least 600° C. Suitable materials are known for use as catalyst supports. They are preferably used in the form of shaped particles having a smallest dimension of at least 0.5 mm. Suitable particles may be in the form of granules, spheres, cylinders, lobed cylinders, rings, saddles or any other shape commonly found as a catalyst support for fixed bed applications. As the skilled person will appreciate, the form and size of the particles affects the heat transfer and pressure drop in a packed reactor bed. The particles must also be sufficiently strong to withstand packing into a reactor and must support the weight of the catalyst bed above without significant breakage. Several suitable particulate materials are known for use in fixed bed catalyst reactors.

The carbon precursor material may be in the form of carbon black, carbon nanotubes, graphene or carbon nanofibers. Such carbons may be active for the dehydrogenation of hydrocarbons without first forming an active carbon phase by contact with the hydrocarbon at elevated temperature, or they may become active after less contact time than the other materials such as alumina.

It has been found by the inventors of the present invention that, at temperatures above about 600° C., certain carbon deposits form on the surface of the catalyst precursor which, it is believed, may be catalytically active in the dehydrogenation of alkanes. The carbon may be graphitic, in the form of graphene layers and/or in the form of nanostructures such as nanofibres or nanotubes. The role of the carbon formed on the catalyst at temperatures greater than 600° C. is not known with certainty. For example, it is possible that the presence of the carbon modifies the surface in a way which is beneficial. For this reason, the invention is not limited to forms in which the carbon formed actively catalyses the dehydrogenation reaction, although it appears likely that the carbon has some catalytic function.

Electron paramagnetic resonance spectroscopy (EPR) is used to detect compounds containing unpaired electrons and the findings indicate that the active carbon species is or contains a radical species. This is believed to be catalytically active, or to contribute towards the catalytic activity of the carbon species. We have found that a carbon species is formed in active catalysts, which, when analysed using EPR, shows a characteristic signal having a peak maximum at a magnetic field strength in the range from 3330-3360 G (Gauss) and a ΔH (line-width) of at least 10 G, more preferably at least 20 G, especially at least 50 G. ΔH is usually <1000, especially <200. ΔH is particularly <100. The line-width represents the difference between the magnetic field strength of the peak maximum and minimum in the characteristic positive and negative pairs of peaks found in such a spectrum and is a measure of the breadth of a peak. The g-value is the magnetic field strength at which the spectrum passes through zero intensity between the positive and negative peaks of the pair. The g-values in this specification refer to EPR spectra measured using the following conditions: frequency 9.47 GHz, power 20 dB (2.2 mW), modulation amplitude 1 G, time constant 20.48 ms, conversion time 40.96 ms. Active catalysts preferably contain carbon having a g-value in the range from 3380-3385 G.

The process includes the step of contacting the hydrocarbon feed with the catalyst precursor at a temperature of at least, and preferably greater than, 600° C., more preferably at least 620° C. We have found that when the temperature is operated at a temperature greater than 620° C., especially greater than 650° C., the conversion and selectivity reach a steady state after about 1-5 hours in which the conversion and selectivity change very little, or increase very slightly during a further period of at least 10 hours. The upper limit of temperature depends on the process economics and the nature of the catalyst precursor, wherein phase changes or sintering may occur if the temperature is raised above a certain point. Normally the process is operated below 850° C. and preferably below 750° C. We have found that, following an initial period of 1-about 6 hours during which the conversion of the hydrocarbon feed falls, the catalyst then maintains its activity and in some cases, increases in activity over periods of several hours so that the requirement for catalyst regeneration is greatly reduced compared with prior art processes. The attainment of “steady state” operation during which both the conversion and yield of dehydrogenated hydrocarbon product remain stable or increase slowly is a feature of the process of the present invention. In the steady state operation of the process the conversion of hydrocarbon feed preferably does not decrease by more than 2% over a period of ten hours.

In a preferred process, the hydrocarbon comprises an alkane which is dehydrogenated to form an unsaturated compound, preferably an alkene. The alkane may be any alkane which is susceptible to dehydrogenation. Linear, cyclic or branched alkanes may be dehydrogenated. Preferred alkanes have from 2 to 24 carbon atoms, especially 3-10 carbon atoms. The dehydrogenation of propane and n-butane are especially preferred reactions because of the commercial importance of their dehydrogenated products, i.e. propene, butenes and butadiene. The hydrocarbon may comprise other compounds which are susceptible to dehydrogenation, in particular compounds containing alkyl substituents such as ethylbenzene, for example.

The feed stream may contain an inert diluent such as nitrogen or another inert gas. Steam may be present in the feed stream. When the process includes a recycle to the reactor, the feed stream may also contain some product compounds such as the alkene(s) formed, hydrogen and any co-products. In one form, the feed stream consists essentially of the reactant hydrocarbon, e.g. an alkane and optionally one or more of an inert gas, and one or more product compounds. Preferably the feed stream does not include more than a trace amount of oxygen. More preferably the process is operated substantially in the absence of oxygen. The process of the invention is not an oxidative dehydrogenation process.

The reactor and/or catalyst bed and/or the feed stream is heated to a temperature sufficient to provide the required reaction temperature. The heating is accomplished by providing heating means of a conventional type known to chemical process engineers.

A portion of the product formed in the process may be recycled to the reactor, with an appropriate heating step if required. The product stream is separated to remove hydrogen, before or after any recycle stream is taken. The products are then further separated into product alkenes and unreacted alkane feed and any by products are removed if required. The process is, however, more selective than some prior art dehydrogenation processes and so the separation train may be greatly reduced compared with that found on a typical prior art dehydrogenation plant, thereby saving on both capital and operating cost. This saving is additional to the reduction in cost realised from the higher conversion and selectivity which is possible using the process of the invention compared with known commercial processes, for example using promoted platinum catalyst at reaction temperatures less than 625° C. For example, known commercial processes typically operate at a conversion of less than 30%. The process of the present invention may be operated at a conversion of 50-60% so that the amount of the feed recycle may be greatly reduced, thus reducing the overall volumetric flow-rate and the associated equipment size.

According to a still further aspect of the invention, we provide a process for the non-oxidative dehydrogenation of a hydrocarbon comprising the step of contacting a feed stream containing at least one hydrocarbon with a catalyst comprising a form of carbon which, which, when analysed using electron paramagnetic resonance spectroscopy (EPR), shows a characteristic signal having a peak maximum at a magnetic field strength in the range from 3330-3360 G and a ΔH (linewidth) of at least 10 G, more preferably at least 20 G, especially at least 50 G. Preferably the g-value (as defined above) is in the range from 3380-3385 G. The catalyst preferably comprises less than 0.1%, especially less than 0.07% of a transition metal selected from the group consisting of V, Cr, Mn, Fe, Co, Mo, Ni, Au, Pt, Pd, Ru and Rh.

According to an alternative aspect of the invention we provide a process for the non-oxidative dehydrogenation of a hydrocarbon comprising the step of contacting a feed stream containing at least one hydrocarbon with a catalyst comprising a form of carbon which is active for the dehydrogenation of alkanes, characterised in that said catalyst comprises
a) a material selected from the group consisting of alumina, silica, magnesia, zirconia, titania, ceria, silica-alumina, carbon and mixtures of these materials, and
b) carbon formed on the surface of said material at an elevated temperature.

The carbon when analysed using electron paramagnetic resonance spectroscopy (EPR), shows a characteristic signal having a peak maximum at a magnetic field strength in the range from 3330-3360 G, a ΔH (linewidth) of at least 10 G, more preferably at least 20 G, especially at least 50 G. The g-value (as defined above) is preferably in the range from 3380-3385 G. The catalyst preferably consists of the material selected from the group consisting of alumina, silica, magnesia, zirconia, titania, ceria, silica-alumina, carbon and mixtures of these materials, and said carbon. The catalyst preferably comprises less than 0.1%, especially less than 0.07%, particularly <0.05% of a transition metal selected from the group consisting of V, Cr, Mn, Fe, Co, Mo, Ni, Au, Pt, Pd, Ru and Rh.

By non-oxidative dehydrogenation, we mean the dehydrogenation of alkanes in the absence of oxygen. In a preferred form of the invention the hydrocarbon comprises at least one alkane and the process is for dehydrogenation of the alkane to form an unsaturated compound, especially an alkene.

### BRIEF DESCRIPTION OF THE DRAWINGS

FIG. 1 (a and b): a plot of dehydrogenation performance vs time for Examples 1-12;

FIG. 2: a plot of propylene yield and dehydrogenation vs time for Example 13;

FIG. 3: a plot of dehydrogenation vs time using carbon black of different bulk density;

FIG. 4: a plot of dehydrogenation vs time using carbon black.

FIG. 5: a plot of dehydrogenation vs time at different starting temperatures using alumina.

FIG. 6: an EPR spectrum of carbon black and alumina following testing.

FIG. 7: an EPR spectrum of alumina following testing at different temperatures.

FIG. 8a-c: EPR spectra of carbon black.

The process will be demonstrated in the following examples and with reference to the accompanying drawings.

### Example 1

### Preparation of Comparative Catalyst A

An aqueous solution of NH₄VO₃ (>99%, Aldrich) was prepared containing oxalic acid to ensure the dissolution of NH₄VO₃ [NH₄VO₃/oxalic acid=0.5 (molar ratio)]. The solution was used to impregnate particles of an extruded theta Al₂O₃ catalyst support in the form of trilobes, using incipient wetness methodology. The solution used was calculated to provide a finished catalyst containing about 3 wt % of vanadium. After impregnation the catalyst precursor was tumbled for 2 h at room temperature to ensure a homogeneous distribution of vanadia on the support. The catalyst was then dried in air at 120° C. overnight and calcined in air for 6 h at 550° C. Analysis of Catalyst A by X-ray fluorescence (XRF) found 3.2% V by weight.

### Dehydrogenation Performance Testing

Dehydrogenation of propane was carried out using a fixed-bed, continuous flow, high temperature stainless steel reactor (1000 mm×18 mm i.d.) connected to an on-line gas chromatography (GC) instrument. The catalyst (9 cm³) was heated (5° C. min⁻¹) to the required reaction temperature in nitrogen (0.5 barg, 160 ml min⁻¹) and held at this temperature to stabilise for at least 30 min. 20% propane (0.5 barg 40 ml min⁻¹) in N₂ was then introduced (total flow 0.5 barg, 200 ml min⁻¹). GC measurements were taken at regular intervals to determine the gas phase composition (propane, propylene, methane, ethane and ethylene). At the end of the run the propane flow was stopped and the catalyst was allowed to cool to room temperature under a flow of N₂ (0.5 barg, 160 ml min⁻¹). The catalyst was taken out of the reactor and the amount of carbon was measured by pyrolysis and infra-red detection using a LECO™ SC-144DR carbon analyser.

The dehydrogenation reaction was run using, as catalyst, the materials listed in Table 1.

The comparative catalyst of Example 1 was tested as described above with the exception that a calcination step was carried out in the reactor prior to the dehydrogenation. The procedure for this was that after the catalyst was loaded into the reactor it was heated (5° C. min⁻¹) to 700° C. in 5% O₂/N₂ (0.5 barg, 140 ml min⁻¹) and held at this temperature for 2 hours. A flow of N₂ (0.5 barg, 160 ml min⁻¹) was then established and the temperature adjusted to the required reaction temperature and held at this temperature to stabilise for at least 30 min. The 20% propane in nitrogen mixture was then introduced into the reactor as described above.

The propane conversion and propylene yield were calculated using the following method:

Propylene yield (%)=100*[propylene out]/[propane in]

In order to account for the propylene produced by thermal cracking of the feedstock, the reaction was repeated with no solid material present in the reactor. The propylene formed at temperatures between 600 and 800° C. was measured. Then, for each dehydrogenation reaction carried out, the amount of propylene which had been formed in an empty reactor at the same reaction temperature was subtracted from the total amount of propylene produced. The dehydrogenation results shown are therefore corrected for the effect of thermal cracking.

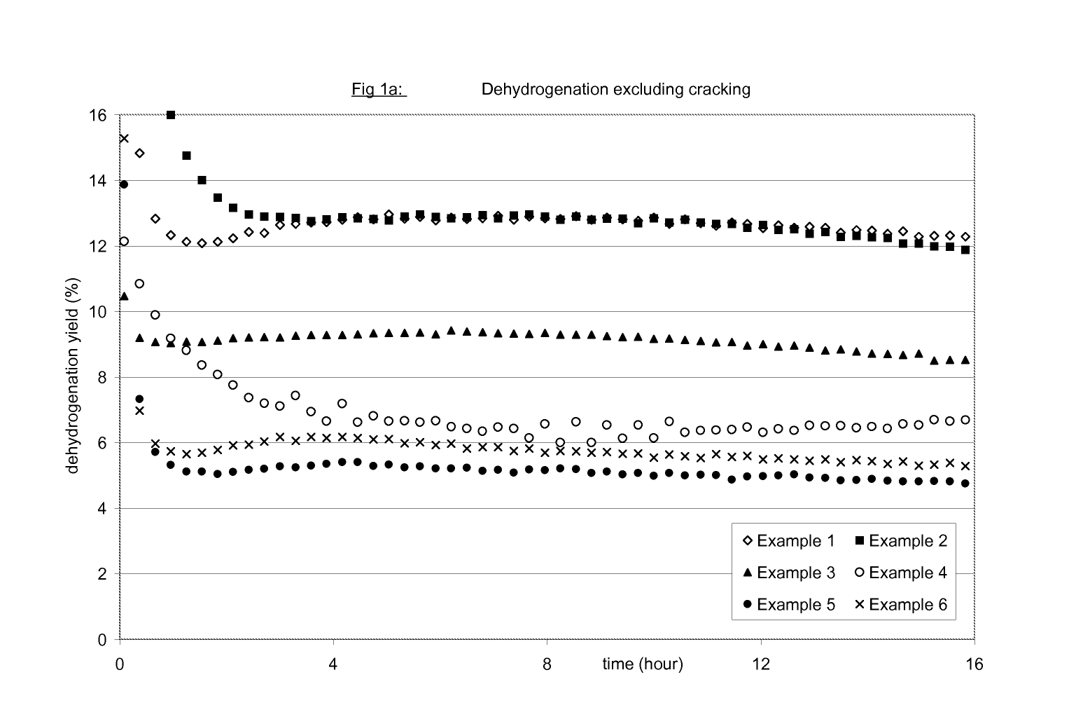

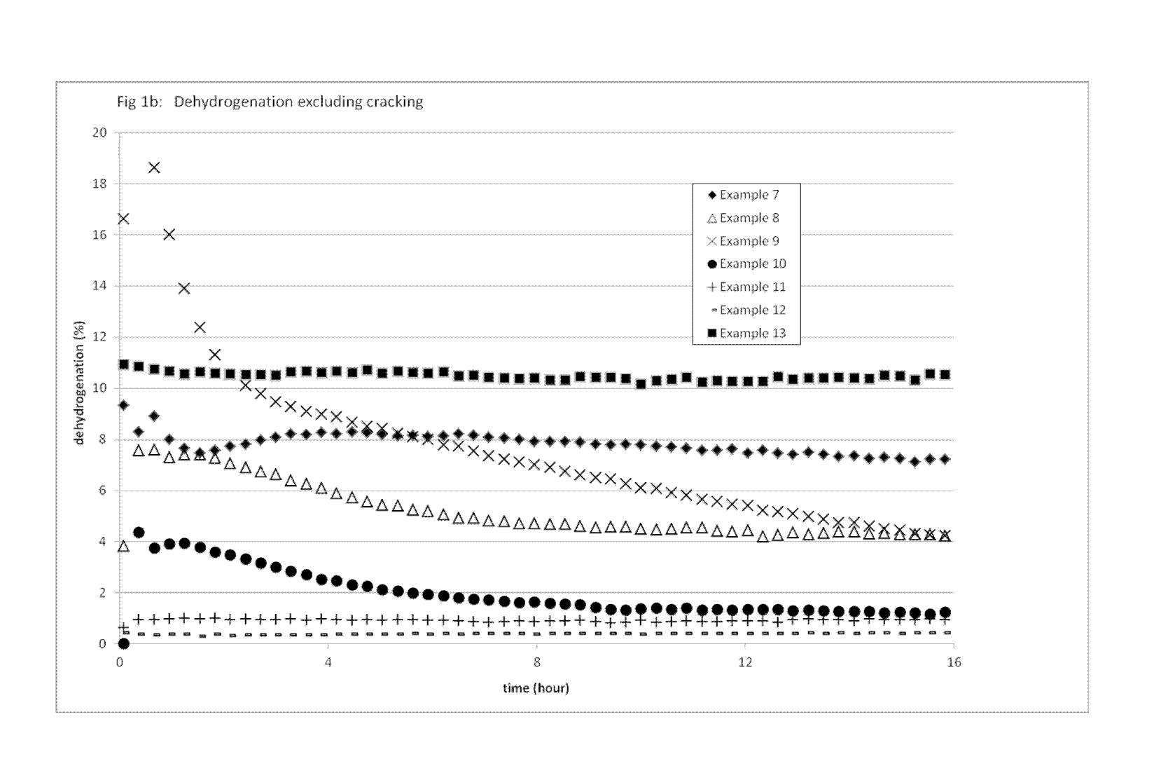

FIG. 1a is a graph showing the catalytic dehydrogenation (i.e. propylene yield−thermal cracking) for Examples 1-6. FIG. 1b shows the same information for Examples 7-12.

TABLE 1

| | | BET | | | Carbon |
|---|---|---|---|---|---|
| | | surface | Propylene | Catalytic | content after |
| | | area | yield | Dehydrogenation | 20 hours |
| Example | Catalyst material | (m²/g) | 15 hours (%) | 15 hours (%) | (wt %) |
| 1 | Catalyst A | | 20.0 | 12.3 | 32.3 |
| | (comparative) | | | | |
| 2 | θ-alumina trilobe | 120 | 21.2 | 12.1 | 37.0 |
| 3 | Carbon black** | 1400 | 17.1 | 8.7 | N/A |
| 4 | Alumino silicate | 440 | 18.9 | 6.5 | 33 |
| | SO275 | | | | |
| 5 | TiO₂ (B) | 40 | 13.9 | 4.8 | |
| 6 | TiO₂ (A) | 146 | 13.5 | 5.3 | 11.6 |
| 7 | ZrO₂ | | 12.5 | 7.2 | |
| 8 | Alumina sphere* | 40 | 14.9 | 4.3 | |
| | SAS-40 | | | | |
| | (α, δ, θ mixed- | | | | |
| | phase) | | | | |
| 9 | Alumina sphere* | 185 | 14.6 | 4.4 | |
| | SAS-200 (γ) | | | | |
| 10 | α-alumina | | 10.0 | 1.2 | |
| 11 | Silicon carbide | | 11.1 | 0.9 | |
| 12 | Silica | | 8.7 | 0.4 | <1 |
| 13 | Carbon | | 16 | 10.5 | N/A |
| | nanotubes*** | | | | |

*sourced from BASF A.G.
**Ketjenblack ™ EC-600JD from Akzo Nobel
***Hyperion Catalysis CS-02D-063-XD

### Example 14

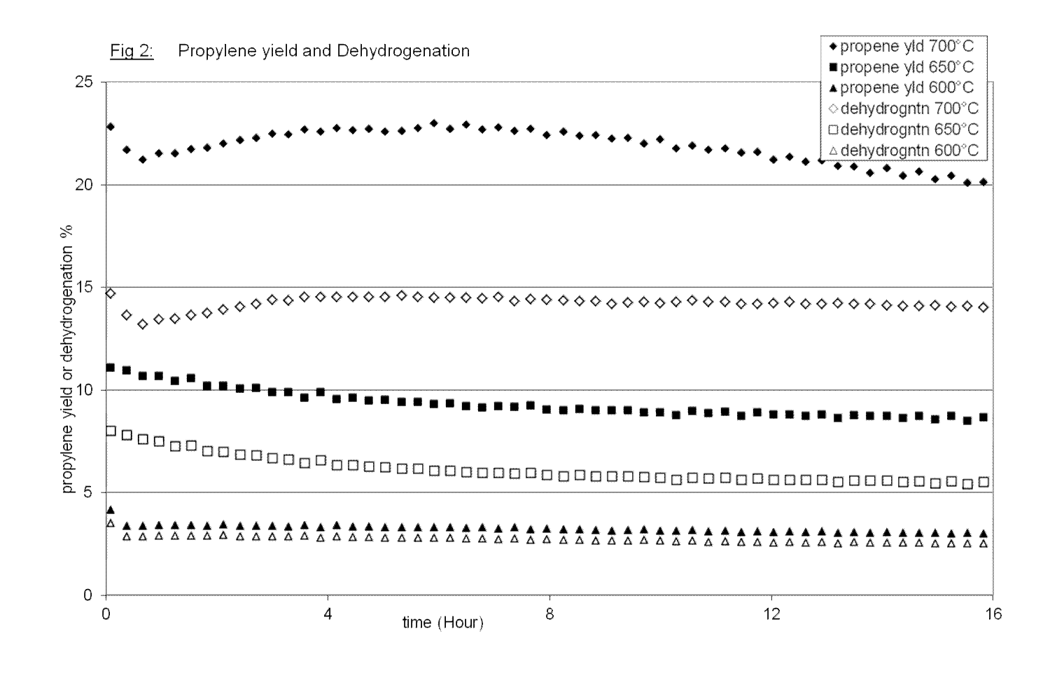

The dehydrogenation experiment was repeated using the carbon black at 600, 650 and 700° C. The results are shown in FIG. 2.

### Example 15

A fresh sample of the carbon black material used in Example 3 was compacted using a semi-automatic Enerpac™ hydraulic pelleting press. Up to three compaction steps, in addition to a pre-compaction, were carried out. The compacted material had the form of cylinders about 5 mm diameter×about 3 mm length. The pressed pellets were fragile especially after only one or two compaction steps. The mass of catalyst used to fill the 9 ml reactor (representative of the bulk density increase with each compaction) is shown in Table 2.

After compaction the material was tested for propane dehydrogenation using the method described in Example 1 (20% propane/80% nitrogen, 9 ml catalyst, 200 ml·min-1 total flow) at 650° C. The rest of the compacted material was ground. The sieved fraction between 200 and 600 microns was used for the next compaction.

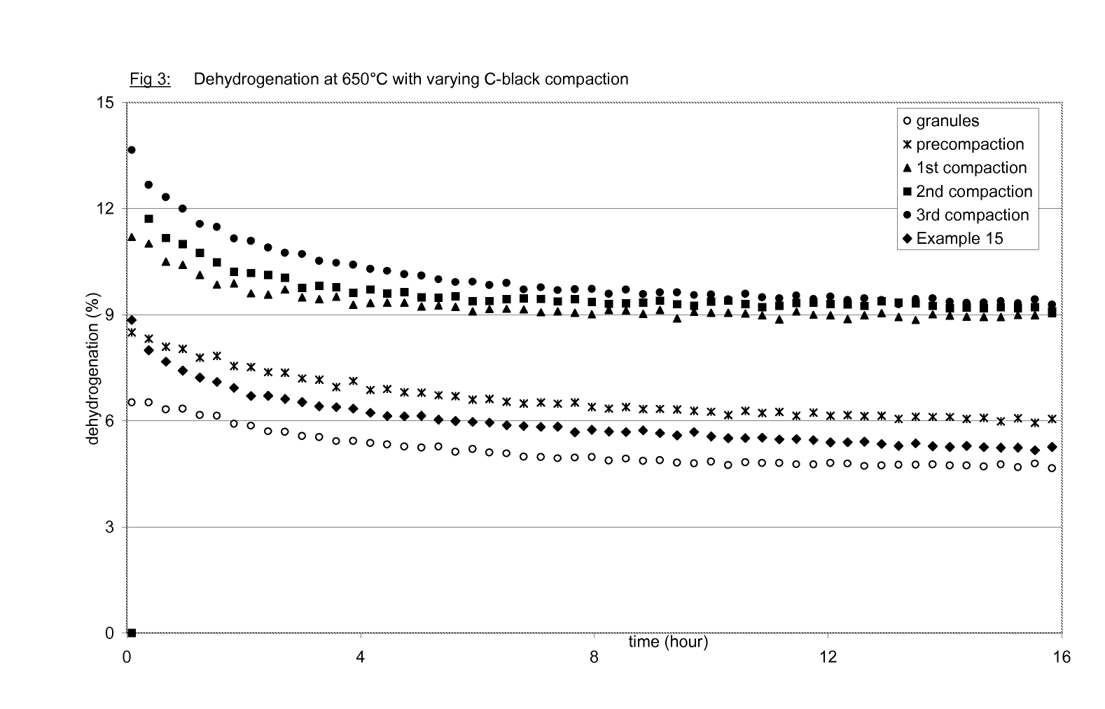

The results of the dehydrogenation experiment, shown in FIG. 3, indicate that the dehydrogenation activity is greatly enhanced by compacting the carbon-black material so that the increased mass of the catalyst within the reactor affects the activity. The increased activity may provide the opportunity to operate the reaction at a lower temperature when using a compacted carbon catalyst. The selectivity to propylene at 15 hours is shown in Table 2 and increases as the carbon is compacted.

TABLE 2

| | Mass of 9 ml | Selectivity 15 h |
|---|---|---|
| | (g) | (%) |
| Granules (as received): | 1.1 | 35 |
| Pre-compaction | 1.7 | 38 |
| 1^{st} press | 1.9 | 52 |
| 2nd press | 2.2 | 54 |
| 3rd press | 2.5 | 59 |

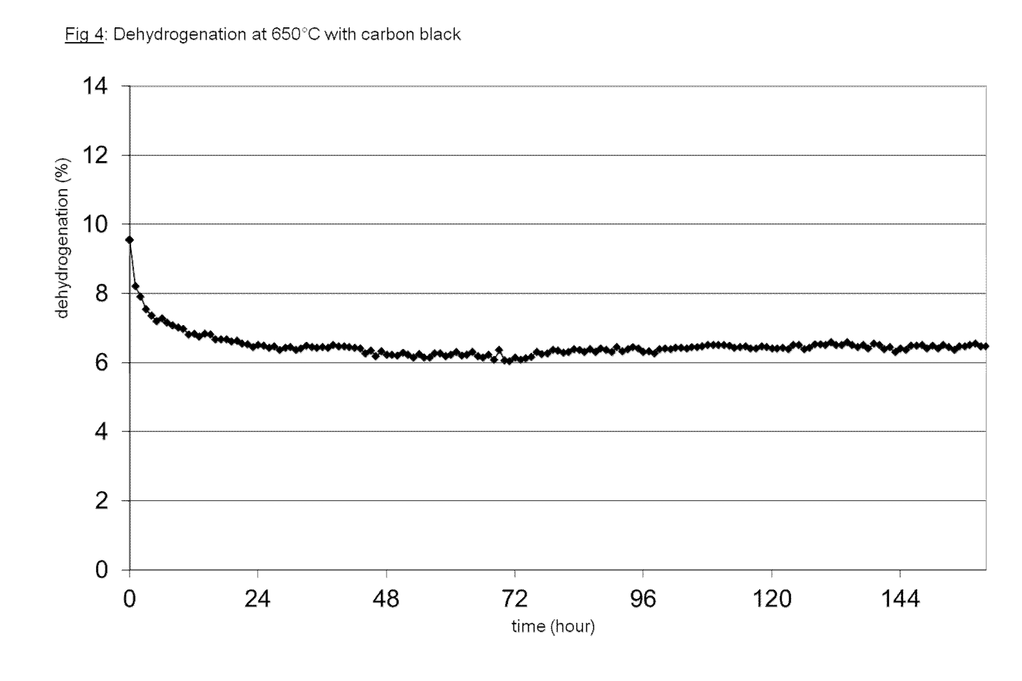

Another sample of the same carbon black material was tested in its pre-compaction form in the same way for about 7 days. The results are shown in FIG. 4 and indicate that the dehydrogenation performance remains stable over this period.

### Example 16

A sample of Vulcan™ XC72R carbon black, supplied by Cabot, was compacted by pre-compaction and a 1^{st} press and tested for propane dehydrogenation using the method described in Example 1. The results are plotted in FIG. 3.

### Example 17

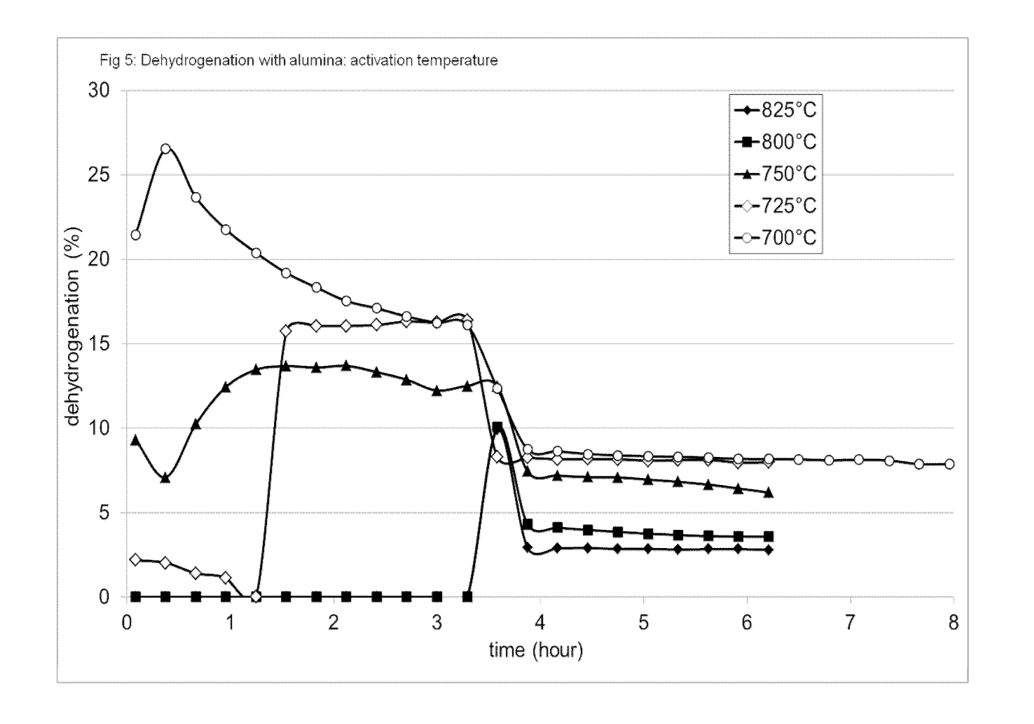

Theta-alumina trilobes, of the type used as a catalyst support and as used in Examples 1 and 2 was tested to determine the effect of different activation temperatures on the dehydrogenation reaction. 9 ml of the trilobes were tested using a reactant stream containing 20% propane as described in the “Dehydrogenation Performance Testing” section above. Each test was carried out using a different starting temperature which was maintained for the initial 3.5 hours of the test following the initiation of the propane flow. After 3.5 hours the temperature was set to 650° C. and the test was continued for up to 8 hours in total. The dehydrogenation activity is shown in FIG. 5. The results indicate that the greatest activity was shown when the test was initially operated at temperatures in the range from 700-725° C. Without wishing to be bound by theory, we believe that the active species of the catalyst, which is believed to be a form of carbon, is formed preferentially formed at 700-725° C. and that, once formed, the catalyst can operate satisfactorily at a lower temperature.

### Example 18

### EPR Analysis of Used Catalysts

Samples of catalyst after use in the dehydrogenation reactions described above were analysed using electron paramagnetic resonance spectroscopy (EPR). EPR is a technique used to identify and characterise free radicals in the compounds studied. The measurements were recorded on a Bruker EMX Micro spectrometer using the following conditions: frequency 9.47 GHz, power 20 dB (2.2 mW), modulation amplitude 1 G, time constant 20.48 ms, conversion time 40.96 ms. Spectra were accumulated with 16 scans.

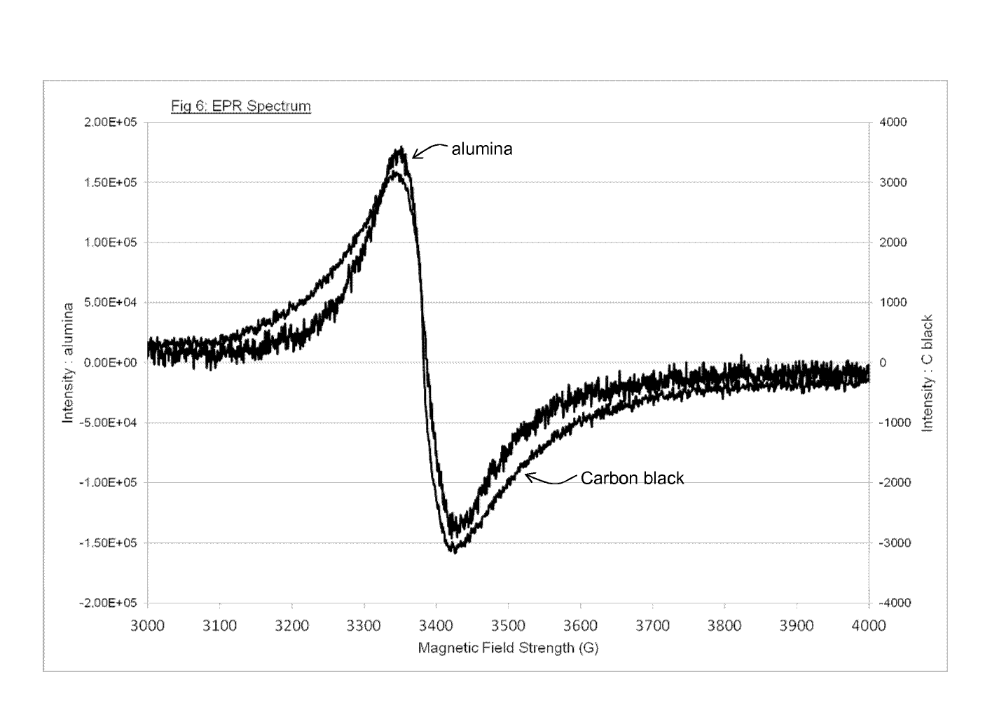

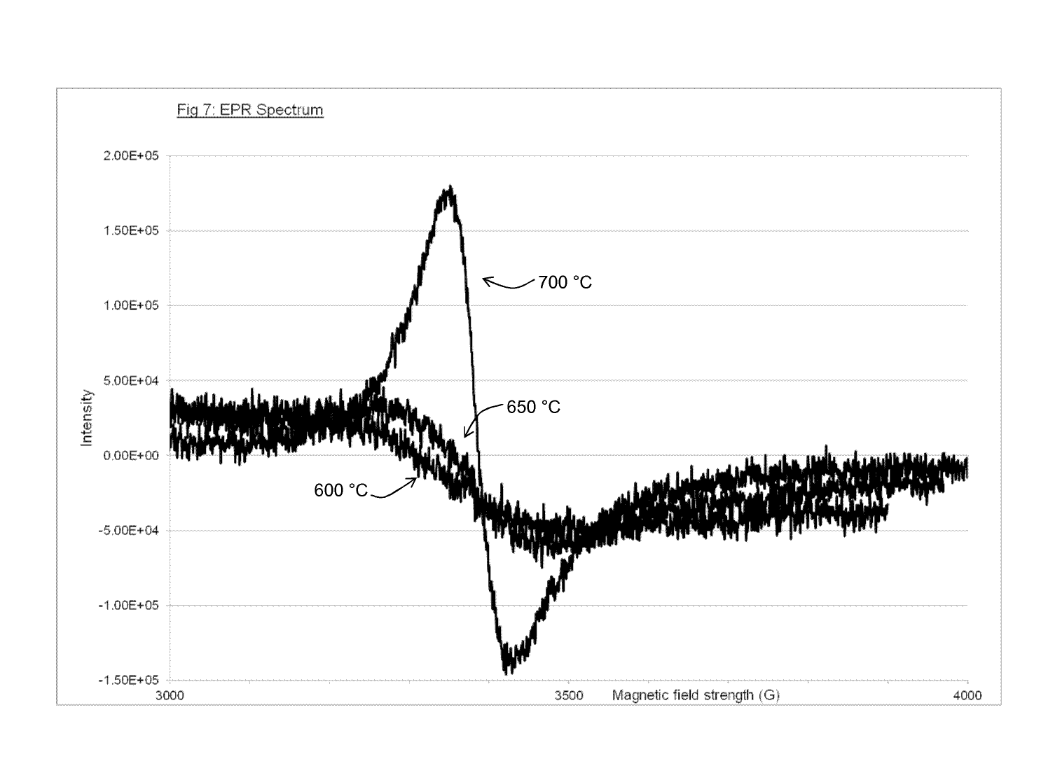

FIG. 6 shows the EPR spectra of used carbon black (Ketjen EC600JD) and used theta Al₂O₃, both having been tested for propane dehydrogenation at 700° C. following procedure described above. The two materials give a similar signal in the EPR spectrum. The alumina signal has a broad EPR signal (peak max at approximately 3351 G) The g value under the conditions used is 3383 G and the ΔH about 80 G. The spectrum of the carbon black has a similar broad peak, having a maximum at about peak max at approximately 3340 G, a ΔH of about 83 G and a g value under the conditions used of 3382 G. The radical species responsible for the signals observed is, we believe, active for the dehydrogenation of propane.

FIG. 7 shows EPR spectra of theta alumina catalysts following their testing for propane dehydrogenation at 600° C., 650° C. and 700° C. respectively, for 20 hours according to the standard procedure. There is a clear difference between the samples, with the broad EPR signal (peak max at approximately 3351 G, ΔH of about 80 G) representing a radical species described above only appearing in the sample tested at 700° C. This is consistent with the results of Example 17 showing enhanced formation of an active species when the alumina material is initially tested or activated at 700-725° C.

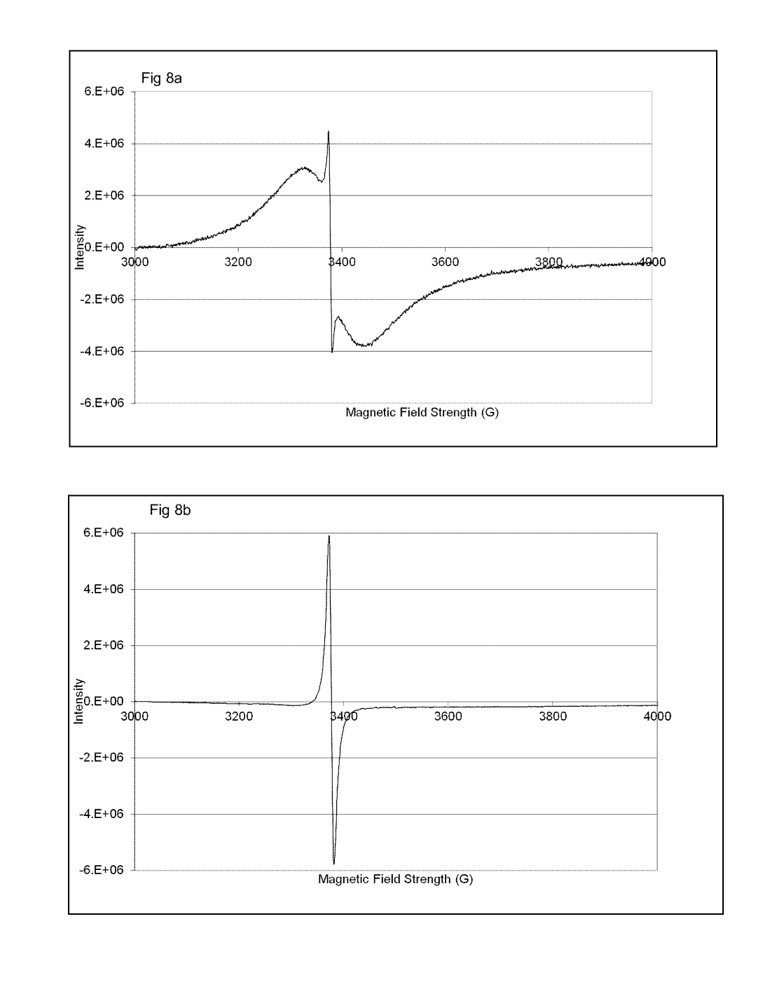

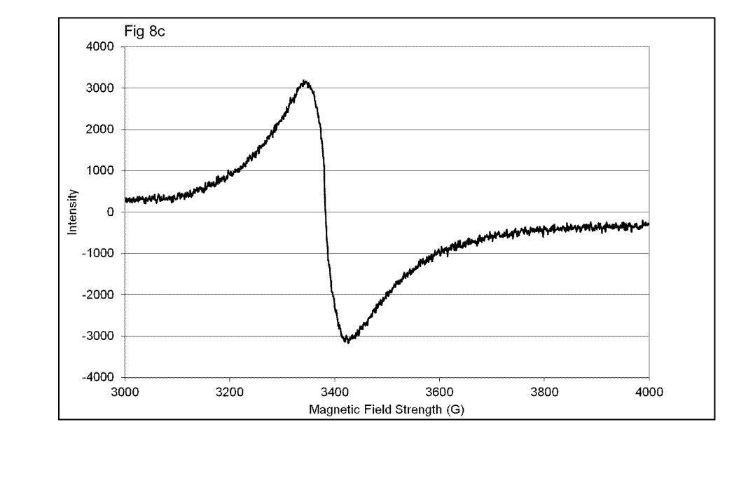

Graphitised carbon black is a sample (of Ketjen EC600JD) which has been treated at 1800° C. in the presence of chlorine. We have found that this material is completely inactive for the dehydrogenation of propane, according to the procedure described above. FIG. 8a and b present the EPR signal for fresh carbon black and fresh graphitised carbon black. The fresh sample of carbon black show two signals by EPR: one broad and one very narrow. The broad signal can be explained by the presence of a particular radical carbon species on Ketjen EC600JD. The graphitisation has removed the radical species responsible for the broad signal while not affecting the radical species responsible for the very narrow signal. The narrow signal is clearly seen in the EPR spectrum at FIG. 8b of the graphitised material, having a peak maximum at 3371 G, a g value under the conditions used of 3377 G and a ΔH of about 9 G. Following testing for propane dehydrogenation, according to our procedure, the radical species responsible for the very narrow signal for non-graphitised carbon black disappears while the radical species responsible for the broader signal remains present, as shown in the EPR spectrum of FIG. 8c (peak max at approximately 3340 G, ΔH of about 83 G). This is the radical species, we believe, is responsible for the dehydrogenation of propane. This is further confirmation that the radical species responsible for a broad signal between 3100 and 3700 is active in the dehydrogenation of propane at 650° C.

1. A process for dehydrogenation of a hydrocarbon comprising the step of contacting a hydrocarbon with a catalyst precursor consisting of a material selected from the group consisting of alumina, silica, magnesia, zirconia, titania, ceria, silica-alumina, carbon black and mixtures of these materials and containing <0.1% by weight of V, Cr, Mn, Fe, Co, Mo, Ni, Au, Pt, Pd, Ru or Rh at a temperature greater than 600° C. for a period of at least an hour.

2. A process according to claim 1, wherein said dehydrogenation reaction is carried out substantially in the absence of oxygen.

3. A process according to claim 1, wherein said elevated temperature is in the range from 650-750° C.

4. A process according to claim 1, wherein said catalyst precursor is activated by contact with said hydrocarbon-containing gas at a temperature between 680 and 750° C. for at least 30 minutes.

5. A process according to claim 4, wherein following said activation, the dehydrogenation is continued at a temperature which is different from the temperature of the activation.

6. A process according to claim 5, wherein following said activation, the dehydrogenation is continued at a temperature which is less than the temperature of the activation.

7. A process according to claim 1, wherein at least 5% of the catalyst, by weight, comprises carbon formed by passing said hydrocarbon-containing gas over said catalyst precursor.

8. A process according to claim 1, wherein said catalyst precursor comprises <0.05% by weight of V, Cr, Mn, Fe, Co, Mo, Ni, Au, Pt, Pd, Ru or Rh.

9. A process according to claim 1, wherein said hydrocarbon comprises an alkane having from 2 to 24 carbon atoms and which is dehydrogenated to form an alkene.

10. A method of forming a catalyst for the dehydrogenation of alkanes, comprising the step of contacting a catalyst precursor consisting of a material selected from the group consisting of alumina, silica, magnesia, zirconia, titania, ceria, silica-alumina, carbon black and mixtures of these materials and containing <0.1% by weight of V, Cr, Mn, Fe, Co, Mo, Ni, Au, Pt, Pd, Ru or Rh with a hydrocarbon at a temperature between 650 and 750° C.

11. A catalyst formed by the method claimed in claim 10.

12. A catalyst consisting of a material selected from the group consisting of alumina, silica, magnesia, zirconia, titania, ceria, silica-alumina, carbon black and mixtures of these materials, and a carbon species, which, when analysed using electron paramagnetic resonance spectroscopy, shows a signal having a peak maximum at a magnetic field strength in the range from 3330-3360 G and a ΔH of >10 G.

13. A catalyst according to claim 12, wherein said electron paramagnetic resonance spectroscopy signal has a ΔH of >50 G.

14. A catalyst according to claim 12 wherein said electron paramagnetic resonance spectroscopy signal has a g-value in the range from 3380-3385 G.

15. A catalyst according to claim 12 containing <0.05% by weight of V, Cr, Mn, Fe, Co, Mo, Ni, Au, Pt, Pd, Ru or Rh.

16. A process according to claim 2, wherein said elevated temperature is in the range from 650-750° C.

17. A process according to claim 2, wherein said catalyst precursor is activated by contact with said hydrocarbon-containing gas at a temperature between 680 and 750° C. for at least 30 minutes.

18. A catalyst according to claim 13, wherein said electron paramagnetic resonance spectroscopy signal has a g-value in the range from 3380-3385 G.

19. A catalyst according to claim 13, containing <0.05% by weight of V, Cr, Mn, Fe, Co, Mo, Ni, Au, Pt, Pd, Ru or Rh.

20. A catalyst according to claim 14, containing <0.05% by weight of V, Cr, Mn, Fe, Co, Mo, Ni, Au, Pt, Pd, Ru or Rh.
